(19) United States
(12) Patent Application Publication
(10) Pub. No.: US 2024/0252395 A1
(43) Pub. Date: Aug. 1, 2024

(54) REMOTE THERAPY PUSH FOR RESPIRATORY THERAPY DEVICES

(71) Applicant: Hill-Rom Services PTE. LTD., Singapore (SG)

(72) Inventors: Justin Xuan Kai Liew, Singapore (SG); Baoyi Wu, Singapore (SG); Leonardo Buenavista Artiaga, JR., Singapore (SG); Soontoong Pua, Singapore (SG); Rayson Ong, Singapore (SG); Wei Bian, Singapore (SG); Stacey Reuland, St. Paul, MN (US)

(21) Appl. No.: 18/427,519

(22) Filed: Jan. 30, 2024

Related U.S. Application Data
(60) Provisional application No. 63/482,446, filed on Jan. 31, 2023.

(51) Int. Cl. A61H 31/00; A61H 9/00

(52) U.S. Cl. CPC A61H 31/00; A61H 9/0007; A61H 9/0078; A61H 2201/165; A61H 2201/5012; A61H 2201/5043; A61H 2201/5058; A61H 2201/5097; A61H 2205/084

(57) ABSTRACT

A therapy devise system includes therapy device with a sensor capable of detecting signals, a control system capable of processing the signals to determine a therapy protocol, and a user interface for assessing and updating the therapy protocol. The control system including a sensor is operable to receive a signal from the server, operable to process the signal to update the therapy device with a new therapy operation protocol. The user interface is operable to display more than one therapy operation protocol and includes a prompt for a user to update the therapy device with the new therapy operation protocol.

### PRIORITY CLAIM

This application claims priority under 35 U.S.C. § 119(e) to U.S. Provisional Application No. 63/482,446, filed Jan. 31, 2023, which is expressly incorporated by reference herein.

### BACKGROUND

The present disclosure relates to the use of systems and methods for communicating with therapy devices being used by a patient in their home, such as a respiratory therapy device. The present disclosure is also directed to systems and methods for automatically updating patient therapy protocols, communicating the updated protocols to the patient, and updating therapy settings in a replacement therapy device.

The use of high frequency chest wall oscillation (HFCWO) techniques are known to provide ongoing pulmonary therapy that may be varied in intensity, frequency, and duration to provide therapy tailored to a particular patient. For example, the Monarch® Airway Clearance System available from Hill-Rom, Inc., Batesville, Indiana, provides mobility with targeted kinetic energy and airflow to thin and mobilize secretions from the airways. The use of such a therapy can be optimized by using images of the lungs/airways to target the provision of therapy to those areas that are in most need of therapy.

Patients use homecare therapy devices including respiratory therapy devices which provide HFCWO therapy on a regular basis as they are going about daily activities. These therapy devices are uploaded with therapy parameters that may need to be updated over the course of patient treatment. Currently, any change in therapy parameters requires the patient to be contacted and guided by an administrator over the phone to change to the required settings. This process is time consuming and prone to human errors. Frequently, the administrator will be required to travel to the patient's home to personally update the therapy parameters. This is not cost effective and increases the risk of disease spreading communicative diseases (e.g., COVID-19, pneumonia). Additionally, it may be difficult to instruct a user to change settings through phone call (language barriers, etc.) or it may be impossible to reach the user via phone.

Accordingly, there is a need in the healthcare field to develop systems and methods for more efficient use of homecare therapy devices. To improve patient health, healthcare costs, time, convenience, and patient's safety, the current disclosure describes systems and methods to remotely and automatically update therapy settings in homecare therapy devices.

### SUMMARY

The present disclosure includes one or more of the features recited in the appended claims and/or the following features which, alone or in any combination, may comprise patentable subject matter.

According to a first aspect of the present disclosure, a remote therapy system for providing therapy to a patient independently of a caregiver, the therapy system includes a remote server, a database, and a therapy device. The database includes one or more therapy operation protocols for the patient, the database operable to communicate the one or more therapy operation protocols with the server. The therapy device is operable by the patient independently of a caregiver to deliver respiratory therapy to the patient while the patient is ambulatory. The therapy device is in communication with the server and programed with a first therapy operation protocol. The therapy device includes a user interface and a control system. The user interface is operable to display the first therapy operation protocol. The control system includes a sensor operable to receive a signal from the server and a controller operable to process the signal to update the therapy device with a second therapy operation protocol. The user interface includes a prompt for the patient to update the therapy device with the second therapy operation protocol.

In some embodiments of the first aspect, the one or more therapy operation protocol is adjusted based on patient training, and wherein the therapy device is operable to communicate with the server and be programmed with an adjusted first therapy operation protocol after the patient training.

In some embodiments of the first aspect, the therapy device is operable to be manually programmed with the adjusted therapy operation protocol after the patient training.

In some embodiments of the first aspect, the one or more therapy operation protocols includes at least one HFCWO therapy parameter and wherein the therapy parameter can include frequency, intensity or duration. In some embodiments of the first aspect, the user interface displays information about the at least one therapy parameter.

In some embodiments of the first aspect, a caregiver remote from the patient and remote from the server can access therapy records communicated to the server by the therapy device after patient use.

In some embodiments of the first aspect, a caregiver remote from the patient and remote from the server can update the server with the second therapy operation protocol over a wi-fi connection. In some embodiments of the first aspect, the server can send the signal to the therapy device after the caregiver updates the server with the second therapy operation protocol.

In some embodiments of the first aspect, the patient can, without intervention by a caregiver, select the first therapy operation protocol or the second therapy operation protocol through the user interface.

In some embodiments of the first aspect, the patient can access logs associated with the first therapy operation protocol or the second therapy operation protocol through the user interface.

In some embodiments of the first aspect, a caregiver remote from the patient and remote from the server can update a default prescribed setting of the patient in the server after the first training session with the user, and wherein the therapy device can access the default setting from the server.

In some embodiments of the first aspect, the user interface includes a basic mode and an advanced mode, wherein the basic mode is updated if the patient selects a standard therapy mode of the user interface and the advanced mode is updated if the patient selects a multiple therapy mode on the user interface.

In some embodiments of the first aspect, the patient can use the user interface to send message to a caregiver remote from the server and the therapy device. In some embodiments of the second aspect, the replacement device can be updated with the one or more therapy protocols on the database by communicating with the server.

According to a second aspect of the present disclosure, a therapy system comprises a remote server, a database, and a high frequency chest wall oscillation therapy device. The database is in communication with the server and includes a therapy operation protocol for a patient. The high frequency chest wall oscillation therapy device is operable by the patient independently of a caregiver to deliver respiratory therapy to the patient while the patient is ambulatory. The therapy device is in communication with the server and includes a user interface and a control system. The user interface is operable to display the first therapy operation protocol. The control system includes a sensor operable to receive a signal from the server and a controller operable to process the signal to update the therapy device with the therapy operation protocol. The therapy device is a replacement therapy device replacing another therapy device being used by the patient. The user interface includes a prompt indicating an update progress to the patient.

In some embodiments of the second aspect, the user interface is configured to prompt the patient when the update is successful.

In some embodiments of the second aspect, the replacement therapy device is operable to be programmed with the one or more therapy operation protocol and the replacement therapy device is operable to communicate with the server. In some embodiments of the second aspect, the replacement therapy device is operable to send therapy records to the server.

In some embodiments of the second aspect, a caregiver remote from the server and the therapy device can access therapy records communicated to the server by the replacement therapy device after patient use through the user interface.

In some embodiments of the second aspect, the patient can select a first therapy operation protocol or a second therapy operation protocol on the user interface of the replacement therapy device.

In some embodiments of the second aspect, the patient can access logs associated with a previous therapy device through the user interface.

According to a third aspect of the present disclosure, a method of using a therapy device operable by a patient independently of a caregiver to deliver respiratory therapy to the patient while the patient is ambulatory, the method including using a control system of the therapy device to receive and process a signal from a server, wherein the signal indicates a therapy operation protocol update for a patient, retrieving a new therapy operation protocol from the server, prompting a user to accept the therapy operation protocol update by accepting a prompt on a user interface on the therapy device, informing the user when the therapy operation protocol update is complete, informing the server when the therapy operation protocol update is complete.

In some embodiments of the third aspect, the method comprises the therapy device receiving the signal when a caregiver inputs a therapy operation protocol update request for the patient.

In some embodiments of the third aspect, the method further comprises requesting feedback from the patient through the user interface after the therapy operation protocol is complete.

In some embodiments of the third aspect, the method further comprises logging all the therapy operation protocol update as reviewable entries through the user interface.

In some embodiments of the third aspect, the method further comprises the user communicating with the caregiver through the user interface.

In some embodiments of the third aspect, the method further comprises the therapy device identifying when a replacement device is required.

In some embodiments of the third aspect, retrieving the new therapy operation protocol from the server is an automated process.

According to a fourth aspect of the present disclosure, a therapy device comprises a user interface and a control system. The user interface is operable to display a first therapy operation protocol. The control system includes a sensor operable to receive a signal from a remote server and a controller operable to process the signal to update the therapy device with a second therapy operation protocol. The user interface includes a prompt for the patient to update the therapy device with the second therapy operation protocol independently of a caregiver.

In some embodiments of the fourth aspect, the therapy device is a high frequency chest wall therapy device that is updated by the patient while the patient is ambulatory and using the therapy device.

In some embodiments of the fourth aspect, the therapy device is operable to communicate with the server and be programmed with an adjusted first therapy operation protocol or an adjusted second therapy operation protocol after patient training.

In some embodiments of the fourth aspect, the therapy device is operable to be manually programmed with the adjusted first therapy operation protocol or the adjusted second therapy operation protocol after the patient training.

In some embodiments of the fourth aspect, the first therapy operation protocol and the second therapy operation protocol include at least one therapy parameter and wherein the therapy parameter can include frequency, intensity or duration.

In some embodiments of the fourth aspect, the user interface includes information about the at least one therapy parameter.

In some embodiments of the fourth aspect, a caregiver remote from the server and the therapy device can access therapy records communicated to the server by the therapy device after patient use.

In some embodiments of the fourth aspect, a caregiver remote from the server and the therapy device can update the server with the second therapy operation protocol over a wi-fi connection. In some embodiments of the fourth aspect, the server can send the signal to the therapy device after the caregiver updates the server with the second therapy operation protocol.

In some embodiments of the fourth aspect, the patient can select the first therapy operation protocol or the second therapy operation protocol through the user interface.

In some embodiments of the fourth aspect, the patient can access logs associated with the first therapy operation protocol or the second therapy operation protocol through the user interface.

In some embodiments of the fourth aspect, a caregiver remote from the server and the therapy device can update a default prescribed setting for the patient in the server after the first training session with the user, and wherein the therapy device can access the default setting from the server.

In some embodiments of the fourth aspect, the user interface includes a basic mode and an advanced mode, wherein the basic mode is updated if the patient selects a standard therapy mode of the user interface and the advanced mode is updated if the patient selects a multiple therapy mode on the user interface.

In some embodiments of the fourth aspect, the patient can use the user interface to send a message to a caregiver remote from the server and the therapy device.

According to a fifth aspect of the present disclosure, a system for remotely modifying operating parameters of a therapy comprises a server configured to receive new operating parameters from a caregiver; a therapy device operable by the patient independently of the caregiver to the therapy to the patient while the patient is ambulatory, the therapy device in communication with the server and programed with existing operating parameters, comprising a user interface operable to display the existing operating parameters and including a prompt for the patient to update the therapy device with the new operating parameters, and a control system including a sensor operable to receive a signal from the server, a controller operable to process the signal to update the therapy device with the new operating parameters if the patient selects the prompt.

In some embodiments of the fifth aspect, the new operating parameters based on patient training, and wherein the therapy device is operable to communicate with the server and be programmed with the new operating parameters after the patient training.

In some embodiments of the fifth aspect, the therapy device is operable to be manually programmed with the new operating parameters after the patient training.

In some embodiments of the fifth aspect, the new operating parameters include at least one high frequency chest wall oscillation (HFCWO) therapy parameter and wherein the HFCWO therapy parameter can include frequency, intensity or duration.

In some embodiments of the fifth aspect, the user interface displays information about the at least one high frequency chest wall oscillation (HFCWO) therapy parameter.

In some embodiments of the fifth aspect, a user remote from the patient and remote from the server can access therapy records communicated to the server by the therapy device after patient use.

In some embodiments of the fifth aspect, the patient can, without intervention by the caregiver, select the existing operating parameters or the new operating parameters through the user interface.

In some embodiments of the fifth aspect, a user remote from the patient and remote from the server can update a default prescribed setting of the patient in the server after a first training session with the patient, and wherein the therapy device can access the default setting from the server.

In some embodiments of the fifth aspect, the user interface includes a basic mode and an advanced mode, wherein the basic mode is updated if the patient selects a standard therapy mode of the user interface and the advanced mode is updated if the patient selects a multiple therapy mode on the user interface.

According to a sixth aspect of the present disclosure, a therapy system comprises a remote server; a database in communication with the server and including a therapy operation protocol for a patient; a high frequency chest wall oscillation therapy device operable by the patient independently of a caregiver to deliver respiratory therapy to the patient while the patient is ambulatory, the therapy device in communication with the server, the therapy device comprising a user interface operable to display the first therapy operation protocol, and a control system including a sensor operable to receive a signal from the server, a controller operable to process the signal to update the therapy device with the therapy operation protocol; and wherein the therapy device is a replacement therapy device, and wherein the user interface includes a prompt indicating an update progress to the patient.

In some embodiments of the sixth aspect, the user interface is configured to prompt the patient when the update is successful.

According to a seventh aspect of the present disclosure, method of using a therapy device operable by a patient independently of a caregiver to deliver respiratory therapy to the patient while the patient is ambulatory, the method comprising the steps of using a control system of the therapy device to receive and process a signal from a server, wherein the signal indicates a therapy operation protocol update for a patient, retrieving a new therapy operation protocol from the server, prompting a user to accept the therapy operation protocol update by accepting a prompt on a user interface on the therapy device, informing the user when the therapy operation protocol update is complete, informing the server when the therapy operation protocol update is complete.

In some embodiments of the seventh aspect, the method comprises the therapy device receiving the signal when a caregiver inputs a therapy operation protocol update request for the patient.

In some embodiments of the seventh aspect, the method further comprises requesting feedback from the patient through the user interface after the therapy operation protocol is complete.

In some embodiments of the seventh aspect, the method further comprises the user communicating with the caregiver through the user interface.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is an illustration of a homecare therapy device embodied as a respiratory therapy device according to the present disclosure;

FIG. 2 is an illustration of a main screen on a user interface in the respiratory therapy device;

FIG. 3 is an illustration of an edit screen on the user interface in the respiratory therapy device;

FIG. 4 is an illustration of a therapy screen on the user interface in the respiratory therapy device;

FIG. 5 is an illustration of connectivity in the respiratory therapy device;

FIG. 6 is an illustration of a method of updating therapy protocols using the respiratory therapy device;

FIG. 7 is an illustration of a successful workflow involved in utilizing the respiratory therapy device for updating therapy protocols;

FIG. 8 is an illustration of a therapy setting update screen on the user interface in the respiratory therapy device;

FIG. 9 is an illustration of a therapy setting screen on the user interface in the respiratory therapy device;

FIG. 10 is an illustration of an unsuccessful workflow involved in utilizing the respiratory therapy device for updating therapy protocols;

FIG. 11 is an illustration of a method of replacing the respiratory therapy device;

FIG. 12 is an illustration of a successful workflow involved in replacing the respiratory therapy device; and

FIG. 13 is an illustration of a respiratory therapy update page on a web portal.

### DETAILED DESCRIPTION

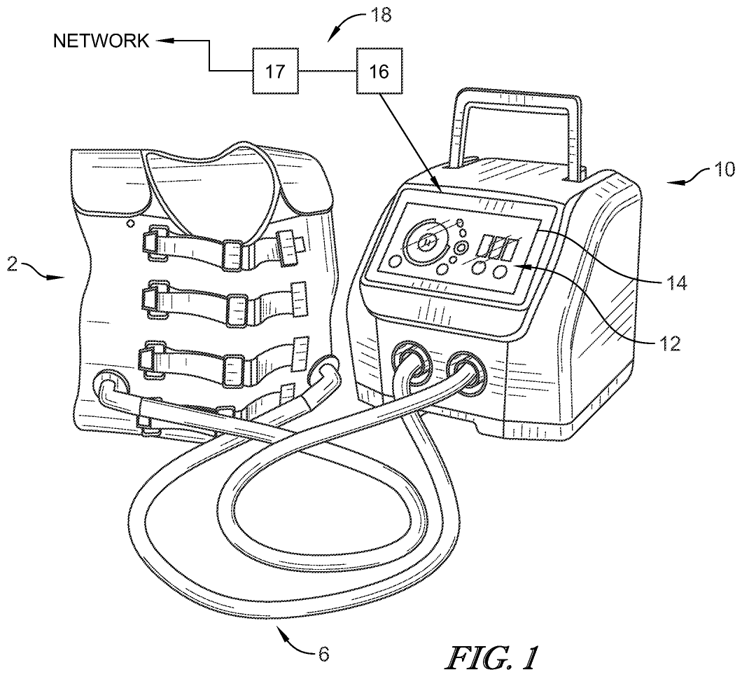

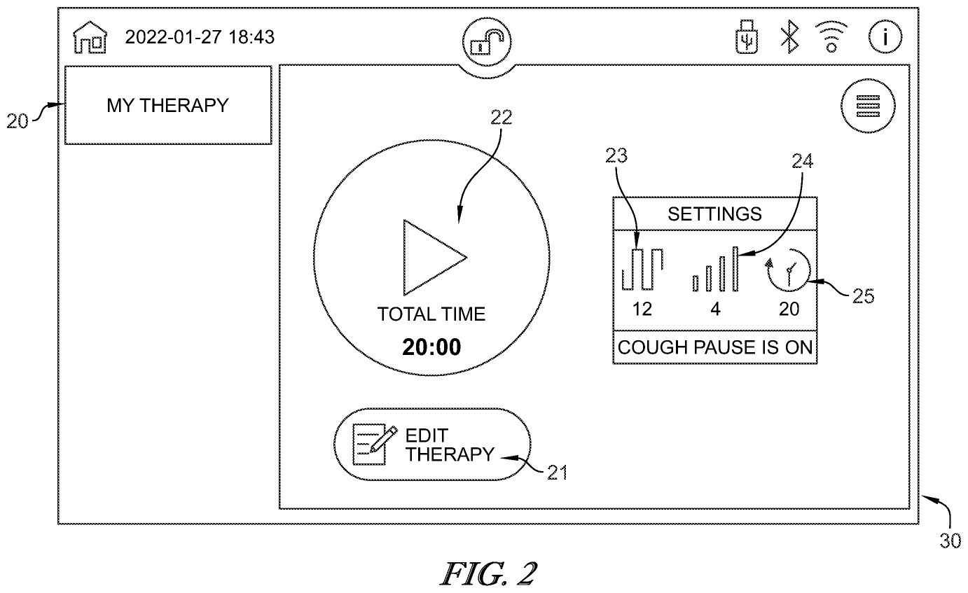

Referring to FIG. 1, a homecare therapy device 10 is illustratively embodied as a respiratory therapy device 10 providing high frequency chest wall oscillation (HFCWO) therapy. The respiratory therapy device 10 includes a user interface 12 including a display 14, and at least one sensor 16 to receive signals over a network. As shown in FIG. 2, the homecare therapy device 10 is configured to be programmed with a therapy protocol 20 including one or more therapy parameters 23, 24, 25, to operate a vest 2 through a connection hose 6. The respiratory therapy device 10 operates as an air pulse generator to inflate the vest 2 during patient therapy as is known in the art. The respiratory therapy device 10 can be used by a patient 50 while the patient is ambulatory and taking part in regular daily activities.

In the illustrative embodiment, the respiratory therapy device 10 includes at the sensor 16 and a controller 17 coupled to the sensor 16 in a control system 18 as shown in FIG. 1. The sensor 16 is configured to receive signals indicative of the therapy protocol 20 for a patient 50 utilizing the respiratory therapy device 10. The controller 17 is configured to process the signals. In one embodiment, the control system 18 is configured to receive the sensor signals, process the sensor signals, and communicate with the patient 50 before changing the therapy protocol 20 programmed on the respiratory therapy device 10. The user interface 12 in the respiratory therapy device 10 can utilize data to illustrate different parameters in the therapy protocol 20.

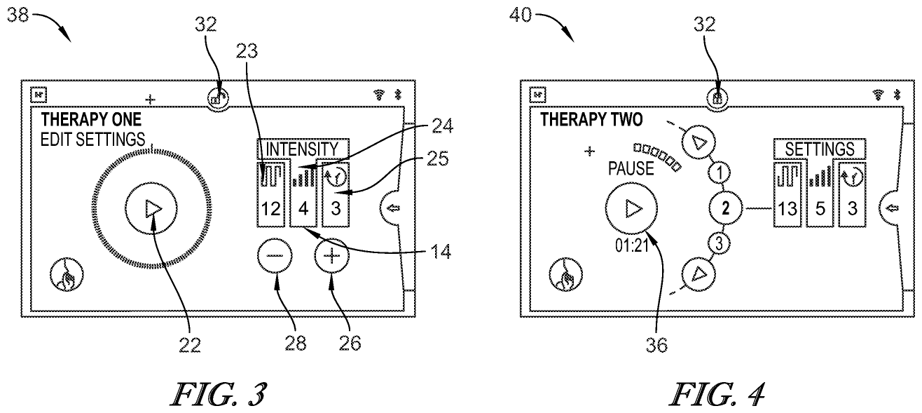

As shown in FIG. 2, the main screen 30 also includes a start button 22 that indicates a total therapy time. An edit button 21 can be used to edit the therapy parameters, frequency 23, intensity 24, and duration 25. As shown in FIG. 3, the patient 50 can edit the parameters on an edit screen 38 upon selecting the edit button 21. The patient 50 can increase or decrease each of those parameters by selecting a plus button 26 or a minus button 28. A cough pause may be included in the protocol therapy based on the duration 25. The cough pause may be for an interval of about 10 seconds to about 1 minute, and can be triggered after the time indicated by the duration 24 has passed. In some embodiments, intensity 24 can range from a level 1 to a level 10. In some embodiments, frequency 23 can range from about 5 Hz to about 20 Hz. In other embodiments, frequency 23 may be more than 20 Hz or less than 5 Hz. In some embodiments, duration 25 can range from about 1 minute to about 60 minutes. In other embodiments, duration 25 may be more than 60 minutes or less than 1 minutes. The patient 50 can select the start button 22 to start the therapy. When the start button 22 is selected, the therapy is locked as indicated by a lock button 32 in FIGS. 3 and 4.

The respiratory therapy device 10 can be operated manually or can be operated based on programmed therapy protocols. The homecare therapy device 10 can include more than one therapy protocol. As shown in FIG. 4, the chosen therapy protocol (e.g., therapy two) can be paused by selecting the pause button 36 on the therapy screen 40.

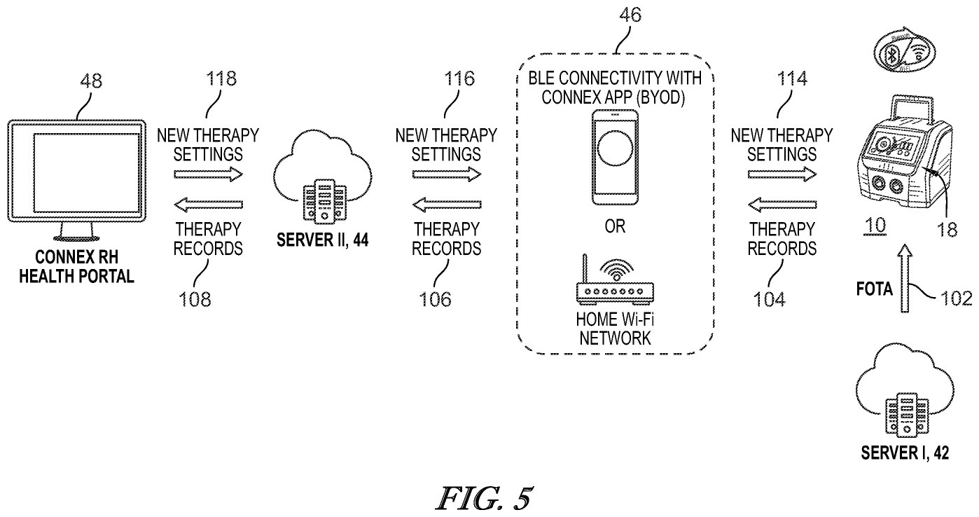

In one embodiment of a system as shown in FIG. 5, the control system 18 is configured to receive a signal from a first remote server 42 in step 102, process the signal, compare and interpret the signal to determine the therapy protocol 20. In the system of FIG. 5, the server 42 and second remote server 44 are remote from both the therapy device 10, patient 50, and a caregiver 52. In this context, a remote server is a server that is not in the same location as the patient 50 or caregiver 52 and, thus, not immediately accessible to the patient 50 or caregiver 52 other than through a communication link When the patient 50 uses the respiratory therapy device 10, therapy records are created and communicated through a home network 46 or a mobile device in step 104, and to second server 44 in step 106. In some embodiments, therapy records are created and communicated through a separate gateway in step 104. The therapy records are further communicated to a health portal 48 in step 108. The health portal 48 can be accessed by a caregiver 52 or an administrator 54. Information, including any new therapy parameters entered into the health portal 48 are communicated from the health portal 46 to the second server 44 in step 118. This new information is further communicated to the home network 46 in step 116, and to the respiratory therapy device 10 in step 114.

The therapy sessions operated through the respiratory therapy device 10 may need to be increased or decreased. Alternatively, or additionally, frequency 23, intensity 24, duration 25, and cough pause setting of any therapy session may need to be altered. The controls system 18 is configured to monitor patient 50 disease progression, titer patient 50 settings as they get accustom to the therapy, identify patients 50 not tolerating therapy due to discomfort, or determine when settings are accidently changed.

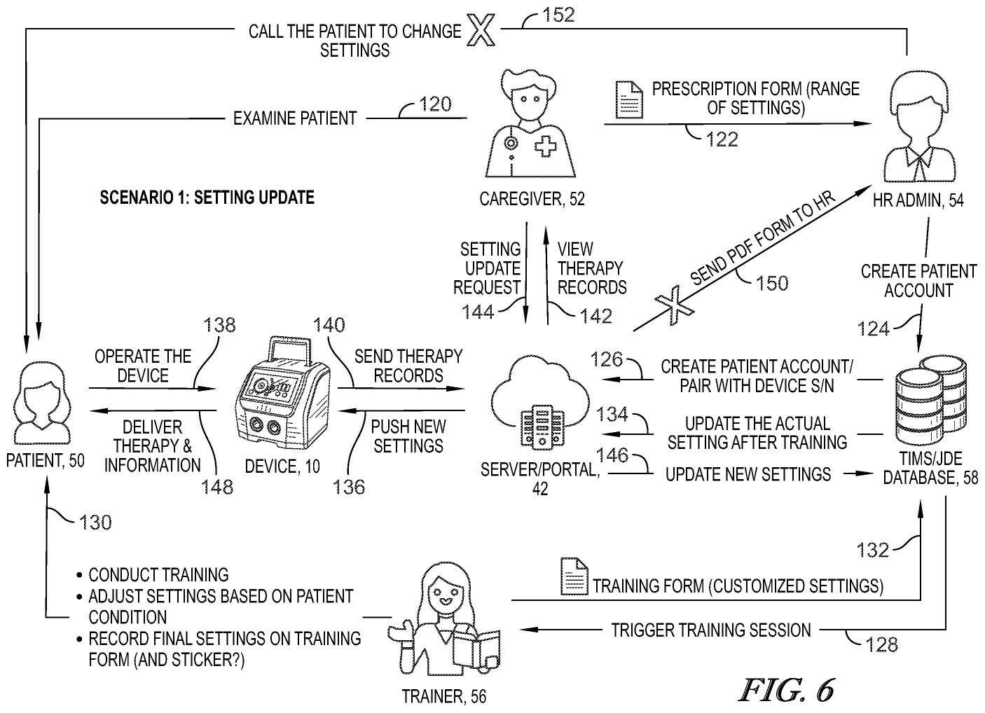

The respiratory therapy device 10 is configured to send and receive instructions from the health portal 48. As shown in FIG. 6, a method of updating therapy protocol using the respiratory therapy device 10 gets rid of the need for the administrator 54 to call the patient 50 to update any settings, thus making a more efficient use of the respiratory therapy device 10. A patient account is created in a database 58 by the administrator 54 in step 124 after the patient 50 is examined for the first time by the caregiver 52 in step 120, and after a prescription form has been sent for the creation of the patient account in step 122. The patient account is paired with the respiratory therapy device 10 through the server 42 in step 126, and the creation of the patient account triggers a training session with a trainer 56 in step 128. The trainer 56 trains the patient 50, adjusts settings based on patient conditions, and records a final setting on a training form in step 130. The updated settings of the therapy protocol 20 are communicated back to the database 58 in step 132. The updated settings are communicated to the server 42 in step 134 and pushed to the respiratory therapy device 10 in step 136.

When the patient operates the respiratory therapy device 10 in step 138, therapy records are sent to the server 42 in step 140, and the caregiver 52 is able to access those therapy records in step 142. If the caregiver 52 determines that the therapy protocol 20 needs to be updated, the caregiver sends an update request to the server 42 in step 144. This request is communicated to the database 58 in step 146 and further pushed to the respiratory therapy device 10 in step 136. The patient 50 accesses the therapy information in step 148. The method described removes at least one of two steps that can decrease efficiency and improve patient health. There is no need of any forms to be sent to the administrator 54 when the caregiver 52 updates the therapy protocol 20 in step 150. Additionally, the administrator 54 does not have to call the patient 50 to notify them of any change in the therapy protocol 20 in step 152. The patient is automatically notified.

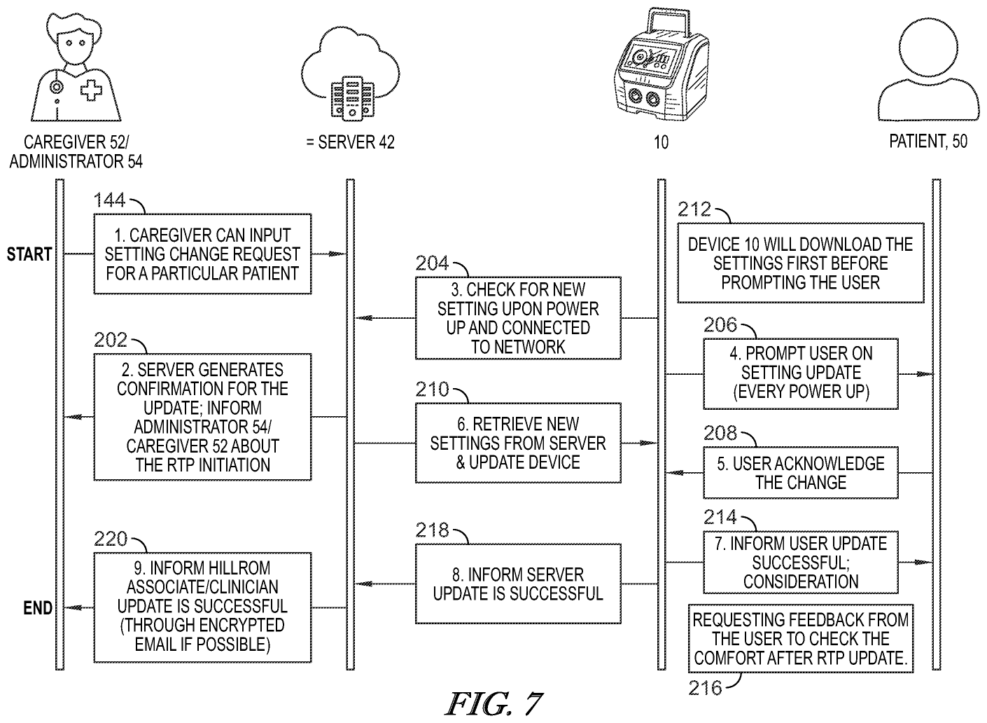

The workflow involved in utilizing the above described process is shown in detail in FIG. 7. When the caregiver 52 inputs a setting change request for a particular patient 50 in step 144, the server 42 generates confirmation for the update; inform administrator 54/caregiver 52 about remote therapy push initiation in step 202. The server 42 is configured to check for new setting inputs every time it is powered up and connected to network in step 204. Once the new settings are communicated to the respiratory therapy device 10 in step 136 identified in FIG. 6, the respiratory therapy device 10 may prompt the patient 50 to update the settings in step 206.

In instances where the respiratory therapy device 10 is not powered up or connected to network for a long time (e.g., due to travel/hospitalization or inability to access network), the caregiver 52 may check request status and/or send caregiver 52 status update periodically. For example, the caregiver 52 may send/check updates on 3^{rd}, 7^{th}, and/or 14^{th} days after the initial update has been communicated by accessing the server 42. If the caregiver 52 makes changes to the settings (i.e., if there is a new request for remote therapy push), the 3^{rd}, 7^{th}, and 14^{th} day reminders can be reset at the server 42. Additionally, or alternatively, if the request may be deemed lapsed after 14 days, and a notification may be sent through the server to the patient 50, caregiver 52, and/or administrator 54.

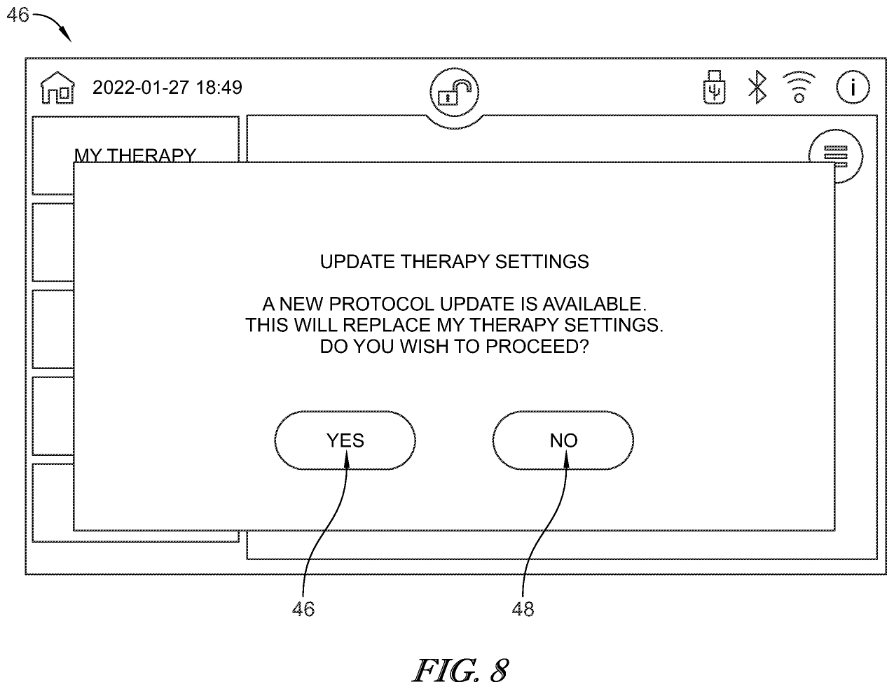

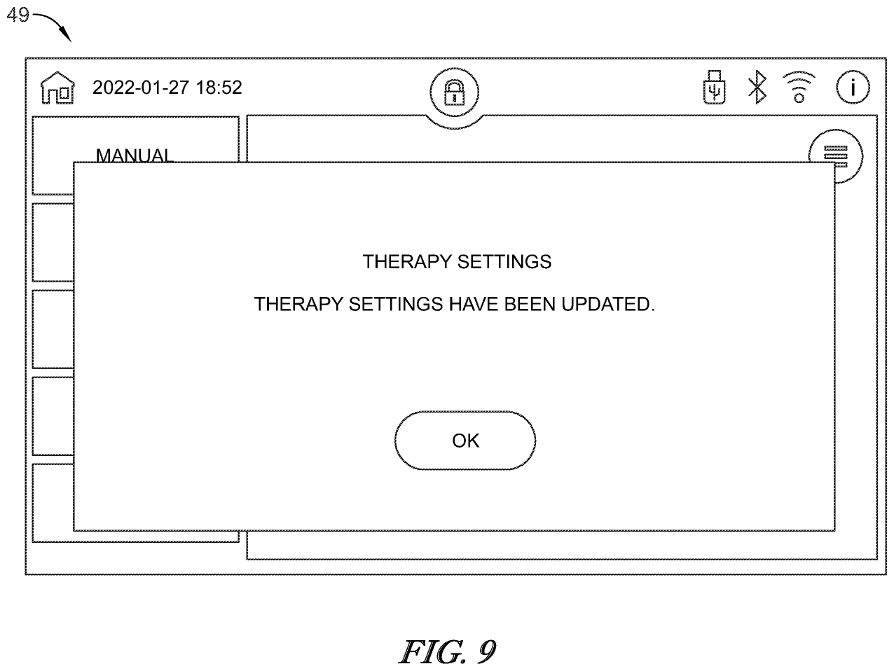

As shown in FIGS. 8-9, the user interface 12 of the respiratory therapy device 10 may require the patient to make affirmative decision to accept any new therapy protocol. A therapy setting update screen 46 may include two choices. The patient 50 can accept the new setting by selecting a yes button 47 or can deny the new setting by selecting a no button 48. If the yes button 47 as shown on a therapy setting screen 49 is selected, the patient 50 acknowledges the change in step 208. In some embodiments, the respiratory therapy device 10 will download the settings first before prompting the patient 50, as shown in step 212.

The patient 50 is notified once the update is successful in step 214. Additionally, feedback may be requested from the patient 50 to check the comfort after remote therapy push update in step 216. Feedback may be limited to a certain number of therapies or days. Feedback may be solicited after a first therapy and a week later. Feedback may be a scaled rating or based on emojis or other symbols. The server 42 is notified once the update is successful in step 218. The caregiver 52 and administrator 54 are notified once the update is successful in step 220. The caregiver 52 and administrator 54 may be notified via email.

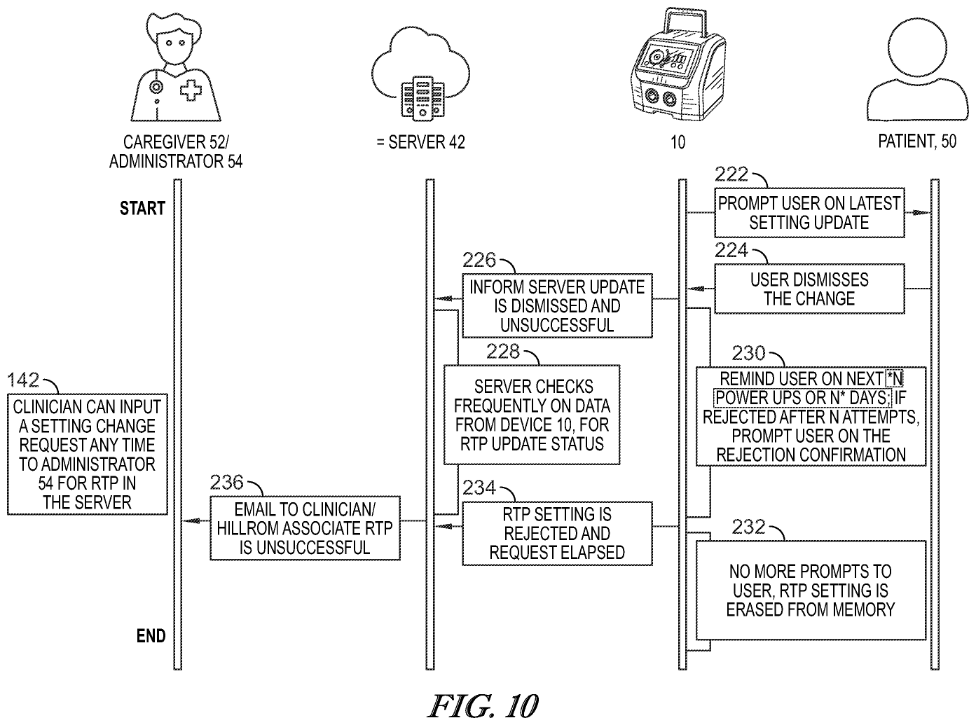

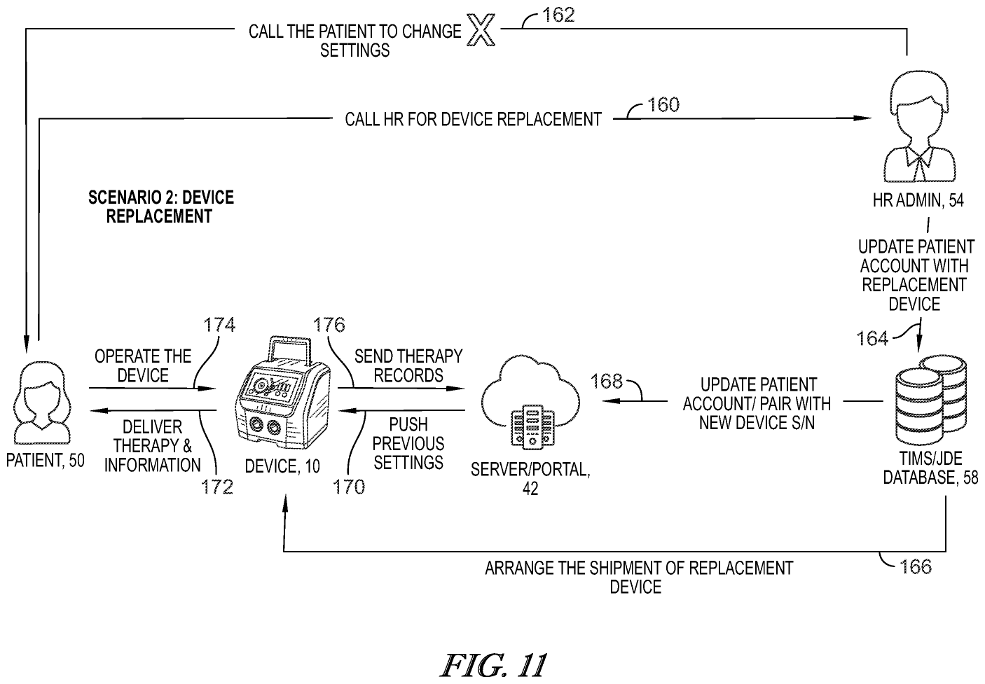

In some embodiments, when the caregiver 52 inputs any changes in the therapy protocol 20, the patient 50 may dismiss it as shown in FIG. 10. Upon receiving a prompt in step 222, as shown in FIGS. 8-10, the patient 50 may dismiss the change in step 224. If the patient 50 dismisses the update in step 226 or selects a “remind me later” feature), the patient 50 may still be allowed to perform therapy with the existing settings. If the therapy protocol update is dismissed by the patient, the server 42 is informed that the update is dismissed and is unsuccessful in step 226. The server 42 may repeatedly check for data from the respiratory therapy device 10 to determine if the patient 50 has accepted the update in step 228.

If the patient 50 rejected the protocol therapy update, the patient 50 is reminded about the therapy protocol update for a fixed number of consecutive power-ups in step 230. For example, the patient 50 may be reminded during 2 to 10 power-ups following the first time an update notification was sent. Alternatively, the patient may be notified or prompted about the protocol therapy update for a fixed number of days. If the protocol therapy update remains rejected after the fixed number of attempts, the patient 50 will be prompted to confirm rejection of the update and remote therapy push setting will be erased from memory of the respiratory therapy device 10 in step 232. A lapse may be requested by the server 42 after rejection by the patient 50. The fact that the remote therapy push request is rejected and the request is deleted is communicated to the server 42 in step 234. An email is sent by the server 42 to the caregiver 52 or administrator 54 notifying them of the failed update attempt in step 236.

The patient may need a replacement respiratory therapy device 10 and may contact the administrator 54 in step 160 as shown in FIG. 11. Upon receiving that message, the administrator 54 updates the patient account in the database 58 with a replacement respiratory therapy device 10 in step 164. The replacement respiratory therapy device 10 is shipped to the patient 50 in step 166. Updated patient account is paired with new device and the information is communicated to the server 42 in step 168. Previous patient settings stored in the old the respiratory therapy device 10 are pushed to the new the respiratory therapy device 10 in step 170. The delivery and therapy information is communicated with the patient 50 in step 172. The patient 50 can operate the new the respiratory therapy device 10 in step 174 and can transmit therapy records from the new the respiratory therapy device 10 to the server 42 in step 176. The administrator 54 does not have to call the patient 50 to change any settings in step 162. This can save crucial time and help improve patient health.

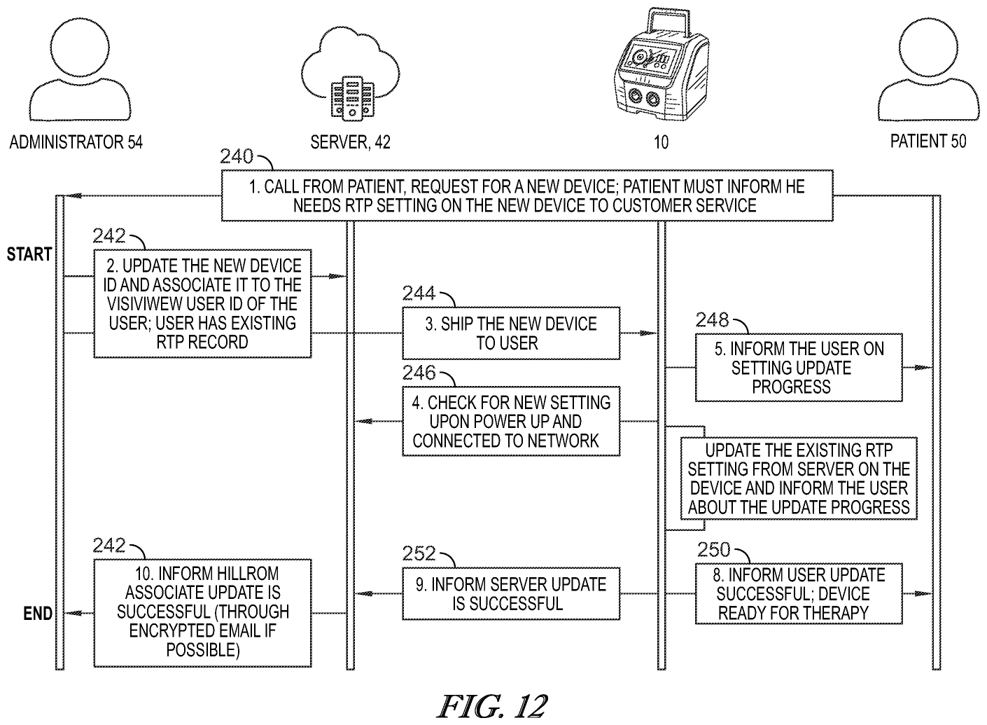

The workflow involved in utilizing the above described process is shown in detail in FIG. 12. When the patient 50 calls the administrator 54 to request a replacement respiratory therapy device 10 in step 240, the patient must inform the administrator 54 that he needs remote therapy push setting on the replacement respiratory therapy device 10. The administrator 54 updates the replacement respiratory therapy device 10 ID and associates it with a user ID of the patient 50 that is linked to an existing remote therapy push record in step 242. The replacement respiratory therapy device 10 is shipped to the patient 50 in step 244. The replacement respiratory therapy device 10 is setup with BYOD or wi-fi connection before being shipped. Once the replacement respiratory therapy device 10 is powered on and connected to the network, it is configured to test for new therapy protocol settings in step 246.

The replacement respiratory therapy device 10 is configured to inform the patient 50 on the progress in settings update in step 248. The existing remote therapy push setting from server 42 is communicated to and updated on the respiratory therapy device 10. The therapy device 10 is configured to download the update progress from the server 42 before informing the patient 50. Thus, the patient 50 is informed about the update progress when the therapy device 10 is powered. If the update is not complete, the protocol setting will continue to be updated once the device is powered. If the update is successful, the patient 50 is notified of the completed update in step 250. The successful completion of the update is communicated to the server 42 in step 252. The successful completion of the update is further communicated to the administrator 54 in step 256. In some embodiments, the update is communicated to the administrator 54 via encrypted email.

If the shipped replacement respiratory therapy device 10 is not setup with Wi-Fi/BYOD connection, the patient 50 can use the default therapy programmed in the replacement respiratory therapy device 10. If the patient 50 did not setup connectivity in the replacement respiratory therapy device 10, remote therapy push is not enabled until the connectivity is enabled. The server 42 may retain the remote therapy push setting for a certain number of days. If the new replacement respiratory therapy device 10 is not updated with remote therapy push and connectivity is not enabled, there will be a follow up call made by the administrator 54. In some embodiments, and an additional proposal (e.g., an email) may be is sent to the patient 50 and/or administrator 54 to enable connectivity and to initiate remote therapy push. A pamphlet or a quick guide about enabling connectivity may be sent to the patient 50.

The user interface 12 may include a provision to update the remote therapy push settings of the patient 50 including but limited to therapy settings, therapy steps, and cough pause settings. The user interface 12 may include a provision to check the latest remote therapy push status of the patient 50 including but limited to a provision for checking the number or frequency of new settings rejected or pending and a provision for checking the number of successful updates.

The user interface 12 may include a provision to log all the remote therapy push updates and activities as reviewable entries. The user interface 12 may include a provision to select a new remote therapy push setting from a list of previously prescribed remote therapy push settings. The user interface 12 may include an email feature to inform the caregiver 52 or administrator 54 about changes to remote therapy push settings on the server 42. The user interface 12 may include an email feature to inform the caregiver 52 or administrator 54 about changes to status of any remote therapy push update from the patient 50. The user interface 12 may include a provision for the trainer 56 or administrator 54 to update a default prescribed setting of the patient 50 in the server 42 after the first training session with the patient 50.

The user interface 12 may include a provision to identify whether the respiratory therapy device 10 is a new device or replacement device. The user interface 12 may include a provision to automate the process of remote therapy push from the server 42 to the respiratory therapy device 10 if there is a replacement device. The user interface 12 may include a provision to update basic or advanced mode settings in the respiratory therapy device 10. For example, if standard or one step is selected, basic mode is selected and updated in the respiratory therapy device 10. Alternatively, if custom mode therapy or multiple therapy steps is selected, then an advance mode is selected and updated in the respiratory therapy device 10.

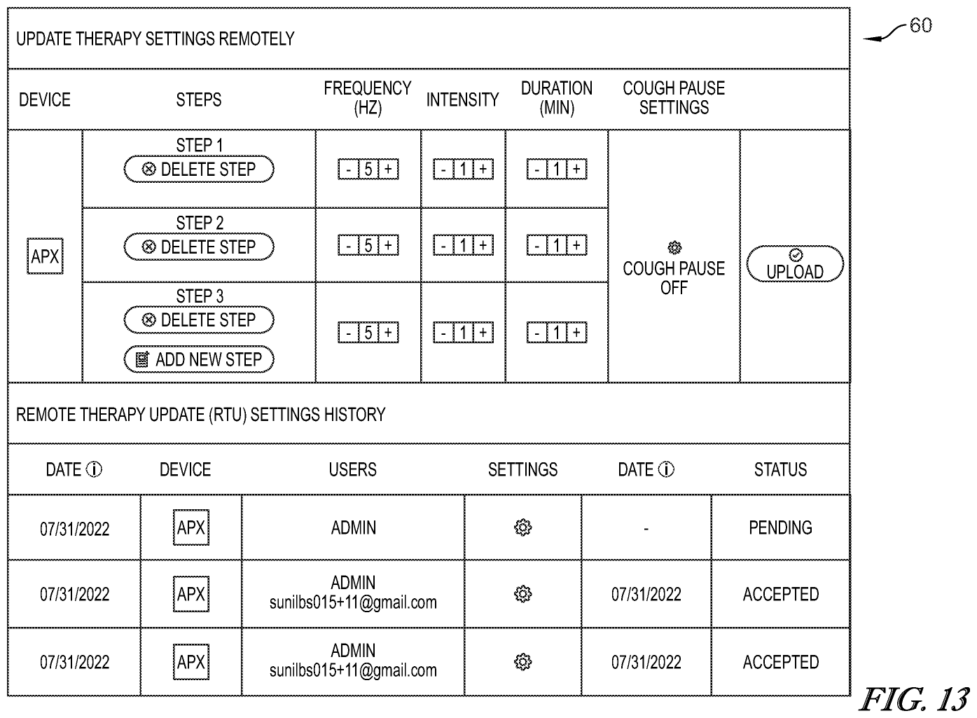

In one embodiment, an app (e.g., Votron) may be used by the patient to access information stored in the server 42. The app may always access the latest respiratory therapy push setting data from the server 42 before sending the respiratory therapy push setting to the patient 50. The app may query for respiratory therapy push settings from server 42 one or more times in one day. Alternatively, or additionally, the app may query for respiratory therapy push settings from server 42 every time the patient logs into the app. FIG. 13 illustrates a respiratory therapy update page 60 that is accessible on a web portal.

In one embodiment, respiratory therapy push setting transfer over Bluetooth low energy (BLE) from the app to the vest 2. In some embodiments, there may be a priority of communication over the several BLE connected devices including the vest 2, a spirometer, and/or other medical devices being utilized by the patient 50. The respiratory therapy push may occur in real time, automatically, or may be user controlled. In some embodiments, the patient 50 may be notified by a pop up message on the user interface 12 of the respiratory therapy device 10 every time there is a respiratory therapy push setting available on the GUI.

Although this disclosure refers to specific embodiments, the disclosure is not limited to the disclosed embodiments. It will be understood by those skilled in the art that various changes in form and detail can be made without departing from the subject matter set forth in the accompanying claims. For example, the disclosure has been illustrated and described in detail in the drawings and the foregoing description, such illustration and description are to be considered illustrative or exemplary and not restrictive. From reading the present disclosure, other modifications will be apparent to a person skilled in the art. Such modifications may involve other features, which are already known in the art and may be used instead of or in addition to features already described herein. In the claims, the word “comprising” does not exclude other elements or steps, and the indefinite article “a” or “an” does not exclude a plurality.

1. A system for remotely modifying operating parameters of a therapy comprising:
a server configured to receive new operating parameters from a caregiver;
a therapy device operable by the patient independently of the caregiver to the therapy to the patient while the patient is ambulatory, the therapy device in communication with the server and programed with existing operating parameters, comprising:
a user interface operable to display the existing operating parameters and including a prompt for the patient to update the therapy device with the new operating parameters, and
a control system including a sensor operable to receive a signal from the server, a controller operable to process the signal to update the therapy device with the new operating parameters if the patient selects the prompt.

2. The system of claim 1, wherein the new operating parameters based on patient training, and wherein the therapy device is operable to communicate with the server and be programmed with the new operating parameters after the patient training.

3. The system of claim 2, wherein the therapy device is operable to be manually programmed with the new operating parameters after the patient training.

4. The y system of claim 1, wherein the new operating parameters include at least one high frequency chest wall oscillation (HFCWO) therapy parameter and wherein the HFCWO therapy parameter can include frequency, intensity or duration.

5. The system of claim 4, wherein the user interface displays information about the at least one high frequency chest wall oscillation (HFCWO) therapy parameter.

6. The system of claim 1, wherein a user remote from the patient and remote from the server can access therapy records communicated to the server by the therapy device after patient use.

7. The system of claim 1, wherein a user remote from the patient and remote from the server can update the server with the new operating parameters over a wi-fi connection.

8. The system of claim 7, wherein the server can send the signal to the therapy device after the caregiver updates the server with the new operating parameters.

9. The system of claim 1, wherein the patient can, without intervention by the caregiver, select the existing operating parameters or the new operating parameters through the user interface.

10. The system of claim 1, wherein the patient can access logs associated with the existing operating parameters or the new operating parameters through the user interface.

11. The system of claim 1, wherein a user remote from the patient and remote from the server can update a default prescribed setting of the patient in the server after a first training session with the patient, and wherein the therapy device can access the default setting from the server.

12. The system of claim 1, wherein the user interface includes a basic mode and an advanced mode, wherein the basic mode is updated if the patient selects a standard therapy mode of the user interface and the advanced mode is updated if the patient selects a multiple therapy mode on the user interface.

13. The system of claim 1, wherein the patient can use the user interface to send message to the caregiver.

14. The system of claim 13, wherein if the patient can request a replacement device from the caregiver, the replacement device can be updated with the new operating parameters by communicating with the server.

15. A therapy system comprising:
a remote server;
a database in communication with the server and including a therapy operation protocol for a patient;
a high frequency chest wall oscillation therapy device operable by the patient independently of a caregiver to deliver respiratory therapy to the patient while the patient is ambulatory, the therapy device in communication with the server, the therapy device comprising:
a user interface operable to display the first therapy operation protocol, and
a control system including a sensor operable to receive a signal from the server, a controller operable to process the signal to update the therapy device with the therapy operation protocol; and
wherein the therapy device is a replacement therapy device, and
wherein the user interface includes a prompt indicating an update progress to the patient.

16. The therapy system of claim 14, wherein the user interface is configured to prompt the patient when the update is successful.

17. A method of using a therapy device operable by a patient independently of a caregiver to deliver respiratory therapy to the patient while the patient is ambulatory, the method comprising the steps of:
using a control system of the therapy device to receive and process a signal from a server, wherein the signal indicates a therapy operation protocol update for a patient,
retrieving a new therapy operation protocol from the server,
prompting a user to accept the therapy operation protocol update by accepting a prompt on a user interface on the therapy device,
informing the user when the therapy operation protocol update is complete,
informing the server when the therapy operation protocol update is complete.

18. The method of claim 17, wherein the method comprises the therapy device receiving the signal when a caregiver inputs a therapy operation protocol update request for the patient.

19. The method of claim 17, further comprising requesting feedback from the patient through the user interface after the therapy operation protocol is complete.

20. The method of claim 17, further comprising the user communicating with the caregiver through the user interface.
